(19) United States
(12) Patent Application Publication
(10) Pub. No.: US 2012/0295818 A1
(43) Pub. Date: Nov. 22, 2012

(54) METHOD OF MULTI PATHOGEN DETECTION

(75) Inventor: Taku Murakami, Irvine, CA (US)

(73) Assignees: HITACHI CHEMICAL RESEARCH CENTER, INC., Irvine, CA (US); HITACHI CHEMICAL CO., LTD., Shinjuku-ku, Tokyo (JP)

(21) Appl. No.: 13/574,270

(22) PCT Filed: Jan. 24, 2011
(86) PCT No.: PCT/US11/00124

§ 371 Date: Jul. 20, 2012

Related U.S. Application Data
(60) Provisional application No. 61/297,616, filed on Jan. 22, 2010.
(60) Provisional application No. 61/301,537, filed on Feb. 4, 2010.

(51) Int. Cl. C12Q 1/04; C40B 30/00; C12Q 1/68; C40B 30/04; C12N 1/20; G01N 33/569

(52) U.S. Cl. 506/9; 435/252.4; 435/7.92; 435/6.12; 435/6.1; 506/7; 435/7.1; 435/34

(57) ABSTRACT

A culturing method includes culturing a sample having one or more species of target pathogens of a detection assay and competing microorganisms under oxygen-poor condition in a growth medium. The growth medium can be a non-selective medium. The oxygen-poor condition is effective to prevent competing microorganisms from suppressing growth of the target pathogens. This method can be used in combination with a sample preparation method for large volume particulate samples using highly porous filter material and porous spherical filter aid.

### FIELD

The present disclosure relates to a method that allows sensitive detection of bacteria of genus Listeria in a sample with competing microorganisms. The present disclosure also relates to a method that allows sensitive detection of multiple species of pathogens including bacteria of genus Listeria, genus Salmonella and genus Escherichia in a sample with competing microorganisms. Further, it also relates to a method that allows sensitive detection of multiple species of pathogens in a large volume particulate sample with a low cost and short hands-on time using highly porous filter and porous spherical filter aid.

### BACKGROUND

In this specification where a document, act or item of knowledge is referred to or discussed, this reference or discussion is not an admission that the document, act or item of knowledge or any combination thereof was at the priority date, publicly available, known to the public, part of common general knowledge, or otherwise constitutes prior art under the applicable statutory provisions; or is known to be relevant to an attempt to solve any problem with which this specification is concerned.

According to a recent estimate by the Centers for Disease Control (CDC), food-borne pathogens cause of 76 million cases of illness, 325,000 hospitalizations, and 5,000 deaths within the United States alone each year. In addition to harmful effects on humans, outbreaks of food-borne illness have detrimental economic effects due to medical costs, lost productivity, product recalls, and halted exports. The USDA recommends a zero-tolerance policy for certain food-borne pathogens, as even a small amount of such pathogens can cause food poisoning or even an outbreak. Therefore, development of sensitive pathogen detection technologies capable of detecting one colony forming unit (cfu) of pathogens in a few grams of food sample is necessary.

Despite many currently available detection technologies and products for food-borne pathogens such as Listeria monocytogenes (L. monocytogenes), Salmonella species, Escherichia coli (E. coli) O157:H7 and Campylobacter jejuni, it is still challenging to detect one cfu of pathogens in a few grams of food sample within a 24 hour time frame. Conventionally, pathogens are extracted from a food sample by dilution and homogenization, which can result in a sample volume of a few hundred milliliters or more. In order to detect small numbers of pathogens in such a large volume of sample, it is necessary to culture the pathogens until their concentration reaches a detection threshold of a suitable pathogen detection assay (pre-enrichment). Time needed for pre-enrichment depends on doubling time, viability of the pathogens, detection threshold, and any growth inhibitory effect of homogenized food samples. Pre-enrichment usually takes at least 12 to 48 hours. Secondary enrichment in more selective growth media may be useful to enrich pathogens of interest more selectively but requires even longer time. Due to the pre-enrichment, even with a highly sensitive detection assay such as polymerase chain reaction (PCR) and enzyme-linked immunosorbent assay (ELISA), detection of low concentration of pathogen contamination in food samples can take one to two days.

L. monocytogenes is one of the major food-borne pathogens and have been responsible for several outbreaks of food-borne illnesses. Conventionally, in order to detect small quantity of L. monocytogenes in food samples, Listeria selective growth medium is used to enrich L. monocytogenes selectively by inhibiting growth of competing microorganisms in same samples. Competing microorganisms having higher starting concentrations and faster growth rates than L. monocytogenes can dominate a culture and inhibit continuous growth of L. monocytogenes in non-selective growth media or weakly selective growth media (the Jameson effect). Presence of competing microorganisms can necessitate secondary enrichment in stronger selective growth medium before concentration of L. monocytogenes can reach the detection threshold.

Selective growth media may contain combinations of selective agents such as antibiotic to inhibit growth of competing microorganisms. Selectivity of selective growth media may be controlled by concentration of the selective agents. One of the disadvantages associated with using selective agents is that many selective agents, especially at higher concentrations, prevent resuscitation of injured pathogens and reduce growth rates of healthy pathogens. Another disadvantage is that when a food sample needs to be tested for multiple species of pathogens, it becomes necessary to aliquot the sample and culture each aliquot in a selective growth medium specific to each species of interest.

In view of the disadvantages of selective growth media, an assay capable of simultaneously detecting multiple pathogen species in a food sample without using selective growth media is desirable. Because of the Jameson effect, enriching multiple pathogens species to a detection threshold of the assay in non-selective growth media is difficult, especially when Listeria species with slow growth rates are of interest and competing microorganisms are present in a sample.

Several growth media such as universal pre-enrichment broth (UPB), No. 17 and an enrichment broth for promoting growth of Salmonella, E. coli and Listeria (SEL), as disclosed in U.S. Pat. No. 5,145,786, U.S. Patent Application Publication No. 2008/0014578 and Appl. Environ. Microbiol. 2008, 74, 4853-4866, respectively, all of which are hereby incorporated by reference in their entirety) have been developed to support simultaneous growth of L. monocytogenes, Salmonella and E. coli O157 from food samples. UPB is highly buffered and low in carbohydrates to prevent rapid pH decreases due to growth of competing microorganisms in culture. Therefore, UPB can support simultaneous enrichment of even injured pathogens. No. 17 has a similar medium composition to UPB. SEL was developed based on buffered Listeria enrichment broth (BLEB) which is a Listeria selective growth medium, by reducing concentrations of antibiotics to support growth of Salmonella and E. coli O157. These growth media have been reported to enrich these pathogens successfully from food samples, however their application in practical food testing has to be further evaluated. There is no report on simultaneous enrichment of Campylobacter species, L. monocytogenes, Salmonella and E. coli O157 so far because enrichment of Campylobacter culture is often done in a microaerophilic environment while L. monocytogenes, Salmonella and E. coli O157 are usually enriched in an aerobic environment.

Described herein is a novel method to simultaneously enrich multiple pathogens species by limiting concentration of dissolved oxygen in non-selective growth media.

While certain aspects of conventional technologies have been discussed to facilitate disclosure of the invention, Applicants in no way disclaim these technical aspects, and it is contemplated that the claimed invention may encompass or include one or more of the conventional technical aspects discussed herein.

### DEFINITIONS

Unless specifically defined herein, all technical and scientific terms have the same meaning as commonly understood by one of ordinary skill in the pertinent art. Also, all publications, patent application publications, and patents identified herein are incorporated by reference in their entirety.

As used herein, “oxygen poor” means a dissolved oxygen concentration that is lower than the oxygen concentration in the ambient environment.

As used herein, “selective agent” or “selective media” means an agent, or media containing one or more agents, that acts to inhibit growth of non-target or competing microorganisms in a culture.

### SUMMARY

The present invention optionally addresses one or more or the problems of deficiencies note above.

Accordingly, the present invention may include a method, such as a culturing method to prevent the Jameson effect, comprising culturing a sample comprising at least one species of a target pathogen and competing microorganisms under an oxygen-poor condition in a growth medium.

According to further aspects, the present invention includes a culturing method to prevent the Jameson effect, comprising culturing a sample comprising at least one target pathogen, and competing microorganisms with higher starting concentrations and/or faster growth rates than the at least one target pathogen, under an oxygen-poor condition in a non-selective growth medium, and conducting a detection assay to detect the presence and/or concentration of the at least one target pathogen.

According to further optional aspects, the present invention may further include one or more, or any combination of one or more, of the following additional aspects and/or steps:

the method(s) described above, wherein the competing microorganisms have faster growth rates and/or higher starting concentrations than the at least one target pathogen;

the method(s) as described above, wherein the growth medium comprises a non-selective growth medium;

the method(s) described above, wherein the competing microorganisms have starting concentrations at least 100 times higher than the target pathogens;

the method(s) described above, wherein the target pathogens comprise one or more of: genus Listeria, genus Salmonella, genus Escherichia and genus Campylobacter,

the method(s) described above, further comprising conducting a detection assay to detect and/or quantify the at least one target pathogen;

the method(s) described above, wherein dissolved oxygen concentration of the growth medium is below 50% of the atmospheric oxygen concentration, optionally, below 25% of atmospheric oxygen concentration, below 10% of atmospheric oxygen concentration, or below 5% of atmospheric oxygen concentration;

the method(s) described above, wherein dissolved oxygen concentration of the growth medium is below 50% of atmospheric oxygen concentration, for example, below 6.6 mg/L;

the method(s) described above, wherein dissolved oxygen concentration of the growth medium is below 25% of atmospheric oxygen concentration, for example, below 3.3 mg/L;

the method(s) described above, wherein dissolved oxygen concentration of the growth medium is below 10% of atmospheric oxygen concentration, for example, below 1.3 mg/L;

the method(s) described above, the growth medium is Brain Heart Infusion Broth, Nutrient Broth or Tryptic Soy Broth;

the method(s) described above, wherein a detection assay is used to detect and/or quantify the target pathogens, the detection assay comprising an agar plate, chromogenic agar plate, enzyme-linked immunosorbent assay (ELISA), immunochromatography, polymerase chain reaction (PCR), reverse transcription PCR(RT-PCR), real-time PCR, real-time RT-PCR, nucleic acid sequence based amplification (NASBA), loop-mediated isothermal amplification (LAMP), isothermal nucleic acid amplification, nucleic acid probe, biosensor, multiplex PCR, multiplex real-time PCR, DNA microarray, protein microarray or Luminex system;

the method(s) described above, wherein the sample has at least two species of target pathogens;

the method(s) described above, wherein the growth medium is a non-selective medium;

the method(s) described above, wherein the non-selective medium is free of antibiotics;

the method(s) described above, wherein the oxygen-poor condition is established by culturing the sample under an atmosphere with less than 20% oxygen, supplementing the growth medium with an oxygen depletion agent, culturing the sample in a seal container, stationary culturing and/or applying a layer of oil on surface of the growth medium;

the method(s) described above, wherein the oxygen depletion agent is Oxyrase enzyme, alcohol oxidase, glucose oxidase, cysteine and/or titanium (III) citrate;

the method(s) described above, the culture medium is a liquid, semi-liquid or solid medium;

the method(s) described above, wherein the sample is located on a highly porous filter material, used to collect the target pathogens and the competing microorganisms from a large volume particulate sample; and/or

the method(s) described above, wherein the at least one target pathogen is a bacteria of genus Listeria.

The present invention may address one or more of the problems and deficiencies of the prior art discussed above. However, it is contemplated that the invention may prove useful in addressing other problems and deficiencies, or provide benefits and advantages, in a number of technical areas. Therefore the claimed invention should not necessarily be construed as being limited to addressing any of the particular problems or deficiencies discussed herein.

### BRIEF DESCRIPTION OF THE DRAWINGS

FIG. 1A shows the growth kinetics of L. monocytogenes in sole culture under aerobic (open circles) and oxygen-poor (closed circles) culture conditions.

FIG. 1B shows the growth kinetics of L. monocytogenes (solid lines) and E. coli O1:K1:H8 (perforated lines) in mixed culture under aerobic (open circles) and oxygen-poor (closed circles) culture conditions.

FIG. 1C shows the growth kinetics of L. monocytogenes (solid lines) and E. coli O1:K1:H8 (perforated lines) in mixed culture under aerobic (open circles) and oxygen-poor (closed circles) culture conditions.

FIG. 2 shows detection assay results of L. monocytogenes in three samples containing L. monocytogenes and E. coli O1:K1:H8, cultured for 24 hours under three different oxygen concentrations.

FIG. 3 shows detection assay results of L. monocytogenes in three samples containing L. monocytogenes and E. coli O1:K1:H8, cultured for 24 hours under another three different oxygen concentrations.

FIG. 4 shows detection assay results of L. monocytogenes in a sample of homogenated ground beef containing L. monocytogenes and E. coli O1:K1:H8, cultured for 24 hours under aerobic and oxygen-poor culture conditions.

FIG. 5 shows detection assay results of L. monocytogenes in samples containing L. monocytogenes, E. coli O157:H7 and E. coli O1:K1:H8, cultured for 24 hours in different growth media, under aerobic and oxygen-poor culture conditions.

FIG. 6 shows a summary of detection assay results of L. monocytogenes in nine samples containing L. monocytogenes and competing microorganisms, cultured for 24 hours under aerobic and oxygen-poor culture conditions.

### DETAILED DESCRIPTION

While the described embodiment represents the preferred embodiment of the present invention, it is to be understood that modifications will occur to those skilled in the art without departing from the spirit of the invention. The scope of the invention is therefore to be determined solely by the appended claims.

According to certain aspects of the present invention, an oxygen-poor culture can effectively prevent the Jameson effect, i.e. a phenomenon that high total microorganism concentration in a culture suppresses growth of all microorganisms therein. Particularly, an oxygen-poor culture can promote continuous growth of L. monocytogenes even in samples with competing microorganisms having high starting concentrations, without use of any selective agents such as antibiotics. In contrast, growth of L. monocytogenes in a sample with competing microorganisms having high starting concentrations is suppressed in aerobic culture due to the Jameson effect, and post-culture concentration of L. monocytogenes fails to reach detection threshold of conventional detection assays. This result was unexpected because oxygen-poor and aerobic conditions did not lead to any difference in growth kinetics of L. monocytogenes in a sole culture (i.e., culture starting with only L. monocytogenes), which is corroborated by several publications. In addition, it was observed that, according to the present invention, oxygen-poor and aerobic conditions did not significantly affect resuscitation of injured L. monocytogenes in sole culture.

A culturing method performed according to the principles of the present invention comprises culturing a sample containing one or more species of target pathogens and competing microorganisms with higher starting concentrations and/or faster growth rates than the target pathogens, under an oxygen-poor condition in a non-selective growth medium, and optionally detecting the target pathogens.

A growth medium generally includes one or more of a carbon source, nitrogen source, amino acids, and various salts for pathogen growth. A growth medium can be a non-selective growth medium to support growth of multiple pathogens such as L. monocytogenes, Salmonella spp. and E. coli O157:H7, and rapid resuscitation of pathogens injured by conditions such as heat, cold, acid, alkali, refrigeration, freeze, pressure and/or vacuum. Preferred growth media for L. monocytogenes, Salmonella spp. and E. coli O157:H7 include Brain Heart Infusion Broth, Nutrient Broth and Tryptic Soy Broth. Alternatively, a growth medium can be a selective growth medium which includes one or more selective agents such as antibiotic against competing microorganisms. However, media with high concentrations of selective agents can prevent resuscitation of injured pathogens and decrease growth rates of all pathogens in a culture. A growth medium can be buffered, preferably with 1 to 500 mM phosphate buffer at pH 5 to 9, more preferably with 10 to 200 mM phosphate buffer at pH 5.5 to 8.5, in order to maintain pH of the growth medium during culture.

An oxygen-poor culture can be conducted according to the present invention in a growth medium with limited dissolved oxygen. The growth medium can be a semi-liquid, solid, or preferably liquid medium. An oxygen-poor condition means that dissolved oxygen concentration in the growth medium during culture is below atmospheric oxygen concentration. More specifically, the dissolved oxygen concentration in the growth medium is under the oxygen-poor condition. Thus, the oxygen concentration can be below 50% of atmospheric oxygen concentration (e.g., 6.6 mg/L or less), alternatively below 25% (e.g., 3.3 mg/L or less), or even below 10% (e.g., 1.3 mg/L or less). Dissolved oxygen concentration can be measured by a commercially available dissolved oxygen sensor such as an Oakton DO300 series meter. Oxygen concentration of a growth medium can be controlled by supplementing the growth medium with an oxygen depletion agent such as Oxyrase enzyme (Oxyrase, Inc., OH), alcohol oxidase, glucose oxidase, cysteine and titanium (III) citrate. For example, the dissolved oxygen concentration of 5 mL BHI medium supplemented with 0, 0.25, 0.5, 1.0, 2.0 or 4.0 g/L L-cysteine was 8.1, 2.4, 0.92, <0.01, <0.01 and <0.01 mg/L, respectively. In another example, the dissolved oxygen concentration of 5 mL BHI broth supplemented with Oxyrase for broth or Oxyrase for agar (Oxyrase, Inc., OH) at 20 or 100 μL/mL was below the detection range of the oxygen sensor, or <0.01 mg/L. The oxygen concentration of a growth medium during culture can also be controlled by methods such as limiting ventilation of oxygen by culturing a sample in a sealed container, culturing a sample in an oxygen-poor atmosphere (e.g., less than 50% oxygen), stationary culture to inhibit oxygen dissolution, and/or applying a layer of oil (e.g. mineral oil) on an interface of a culture and atmosphere to inhibit oxygen dissolution. It can be also useful to monitor dissolved oxygen concentration of a growth medium during culture directly or indirectly for quality control. Indirect measurement of dissolved oxygen concentration can be done by measuring gaseous oxygen concentration above a culture in a container. A disposable oxygen sensor such as Red Eye Oxygen sensor patch (Ocean Optics, FL) can be useful to monitor oxygen concentration of an oxygen-poor culture in a non-invasive manner.

The method described herein has a significant advantage. It allows simultaneous culture of multiple species of target pathogens in a sample (such as species in genus Listeria (e.g. L. monocytogenes), genus Salmonella, genus Escherichia and/or genus Campylobacter), in presence of competing microorganisms (such as E. coli, Salmonella or other microorganisms frequently found in food samples) with higher starting concentrations and/or faster growth rates than the target pathogens. Such competing microorganisms in conventional aerobic cultures suppress growth of the target pathogens due to the Jameson effect. In contrast, the oxygen-poor condition in the method of the present invention promotes growth of the target pathogens despite the presence of competing microorganisms and precludes the Jameson effect.

Detection assays can be employed to detect the target pathogens in a culture conducted as described herein when the target pathogens reach the detection threshold levels of the detection assays. Multiple species of the target pathogens can be detected separately or simultaneously. A detection assay can include extracting and detecting characteristic cellular components of the target pathogens such as genomic DNA, ribosomal RNA, transfer RNA, messenger RNA, or protein. Characteristic cellular components can be extracted by using standard molecular biology techniques or commercial products, and detected by conventional techniques such as agar plate, chromogenic agar plate, enzyme-linked immunosorbent assay (ELISA), immunochromatography, polymerase chain reaction (PCR), reverse transcription PCR(RT-PCR), real-time PCR, real-time RT-PCR, nucleic acid sequence based amplification (NASBA), loop-mediated isothermal amplification (LAMP), any other isothermal nucleic acid amplification, nucleic acid probe and/or biosensor. In order to detect multiple species of the target pathogens separately, the culture or the extracted characteristic cellular components can be split into aliquots and each of which is tested by a detection assay specific to a species of the target pathogens. In order to detect multiple species of the target pathogens simultaneously, the culture or the extracted characteristic cellular components can be tested by a conventional multiplex pathogen detection assay such as multiplex PCR, multiplex real-time PCR, DNA microarray, protein microarray and Luminex system.

Methods of the present invention can be used in combination with a sample preparation method for large volume particulate samples using highly porous filter material and porous spherical filter aid, as disclosed in commonly assigned PCT publication WO2009018544, which is hereby incorporated by reference in its entirety. Specifically, a large volume particulate sample such as food homogenate and an environmental sample can be filtered by a highly porous filter material in which pathogens in the sample can be immobilized. The filter material can be cultured in a growth medium with limited dissolved oxygen concentration as described above (i.e. under an oxygen-poor culture condition). Optimal volume of the growth medium depends on sizes of the filter material. Minimum amount growth medium sufficient to support growth of the immobilized pathogens is preferably used in order to obtain maximum pathogen concentration. For example, less than 5 mL for a filter material with 47-mm diameter or less than 10 mL for a filter material with 70-mm diameter is preferable. At the end of the culture, target pathogens can be collected from the growth medium. In order to maximize recovery of the target pathogens, the filter material can be torn, shredded, broken, or cut into small pieces before, during or after the culture in order to promote extraction of the target pathogens from the filter material and/or growth of the target pathogens. The target pathogens in the growth medium can be detected using assays described hereinabove.

### Example 1

Growth kinetics of L. monocytogenes in aerobic and oxygen-poor conditions in absence and presence of competing microorganisms (E. coli) were investigated, respectively.

Sole Cultures of L. monocytogenes.

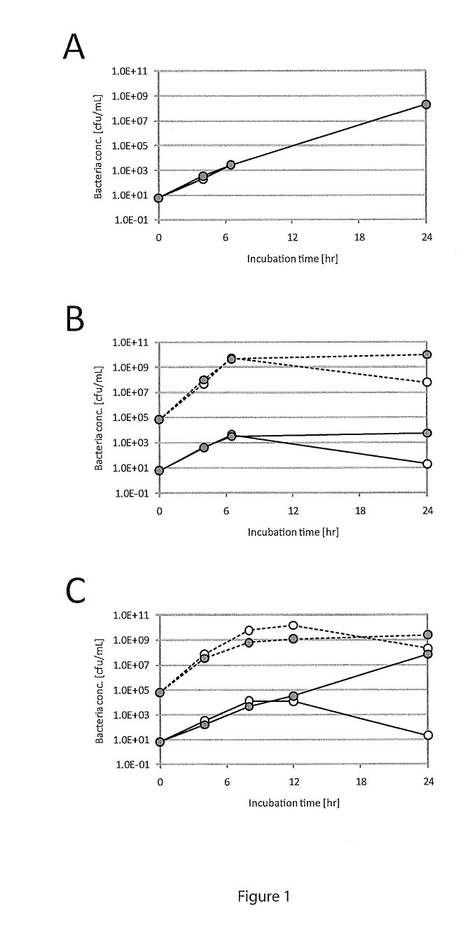

Two 5 mL Brain Heart Infusion Broth samples inoculated with 6.1 cfu/mL L. monocytogenes 4b were cultured on a rotational shaker (250 rpm) at 37° C. under aerobic and oxygen-poor conditions, respectively. The aerobic condition was established by allowing air ventilation during culture, and the oxygen-poor condition was established by preventing air ventilation. L. monocytogenes was detected by plate counting at several culture time points up to 24 hours to construct growth curves of L. monocytogenes. As shown in FIG. 1A, no significant difference was observed in growth kinetics of L. monocytogenes between the samples cultured under the aerobic (open circles) and the oxygen-poor (closed circles) conditions.

Co-Cultures of L. monocytogenes and Competing Microorganisms.

Two 5 mL Brain Heart Infusion Broth samples inoculated with 6.1 cfu/mL L. monocytogenes 4b and 8.3×10⁴ cfu/mL E. coli O1:K1:H8 were cultured on a rotational shaker (250 rpm) at 37° C. under the aerobic and oxygen-poor conditions as done in the sole cultures, respectively. L. monocytogenes and E. coli O1:K1:H8 were detected by plate counting at several culture time points up to 24 hours to construct growth curves of L. monocytogenes (solid lines) and E. coli O1:K1:H8 (dotted lines). As shown in FIG. 1B, growth of L. monocytogenes was significantly promoted in the sample cultured under the oxygen-poor condition (closed circles) compared to in the sample cultured under the aerobic condition (open circles).

Co-Cultures of L. monocytogenes and Competing Microorganisms.

Two 5 mL Brain Heart Infusion Broth samples inoculated with 6.8 cfu/mL L. monocytogenes and 6.4×10⁴ cfu/mL E. coli O1:K1:H8 were cultured at 37° C. under the aerobic and other oxygen-poor conditions as was done in the sole cultures described above, respectively. Aerobic condition was created by allowing air ventilation through rotational incubation (250 rpm), while oxygen-poor condition was created by preventing air ventilation through stationary incubation. L. monocytogenes and E. coli O1:K1:H8 were detected by plate counting at several incubation time points up to 24 hours to obtain growth curves of L. monocytogenes (solid lines) and E. coli O1:K1:H8 (dotted lines) (FIG. 1C). Growth of L. monocytogenes was significantly promoted by oxygen-poor incubation (closed circles) comparing aerobic incubation (open circles).

### Example 2

### Effect of Dissolved Oxygen Concentrations in Growth Media on Growth of L. monocytogenes in Co-Culture with Competing Microorganisms

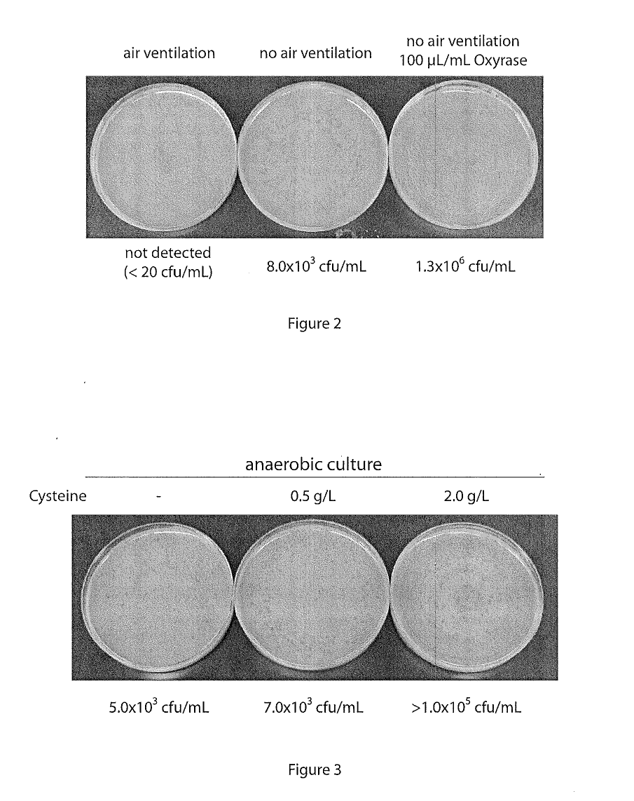

In a first set of experiments, three 5 mL Brain Heart Infusion Broth samples inoculated with 1 cfu/mL L. monocytogenes 4b and 1×10⁵ cfu/mL E. coli O1:K1:H8 were cultured in a rotational shaker (250 rpm) at 37° C. at different dissolved oxygen concentrations established by (from high to low): air ventilation, no air ventilation, and no air ventilation with 100 μL/mL Oxyrase for agar (Oxyrase, OH), respectively. The dissolved oxygen concentrations were estimated at 8.7 mg/L for air ventilation, 8.1 mg/L for no air ventilation, and <0.01 mg/L for no air ventilation with 100 μL/mL Oxyrase for agar. After 24-hour culture, L. monocytogenes was detected and quantified by plate counting. As shown in FIG. 2, growth of L. monocytogenes was significantly promoted by reducing dissolved oxygen concentration.

In a second set of experiments, three 5 mL Brain Heart Infusion Broth samples inoculated with 6.0 cfu/mL L. monocytogenes 4b and 7.0×10⁴ cfu/mL E. coli O1:K1:H8 were cultured in a rotational shaker (250 rpm) at 37° C. at different dissolved oxygen concentrations established by (from high to low): no air ventilation, no air ventilation with 0.5 g/L L-cysteine, and no air ventilation with 2.0 g/L L-cysteine, respectively. Dissolved oxygen concentrations were estimated at 8.1 mg/L for no air ventilation, 0.92 mg/L for no air ventilation with 0.5 g/L L-cysteine and <0.01 mg/L for no air ventilation with 2.0 g/L L-cysteine. After 24-hour culture, L. monocytogenes was detected and quantified by plate counting. As shown in FIG. 3, growth of L. monocytogenes was significantly promoted by reducing dissolved oxygen concentration.

### Example 3

### Co-Cultures of L. monocytogenes and Competing Microorganisms in 10% Ground Beef Homogenates

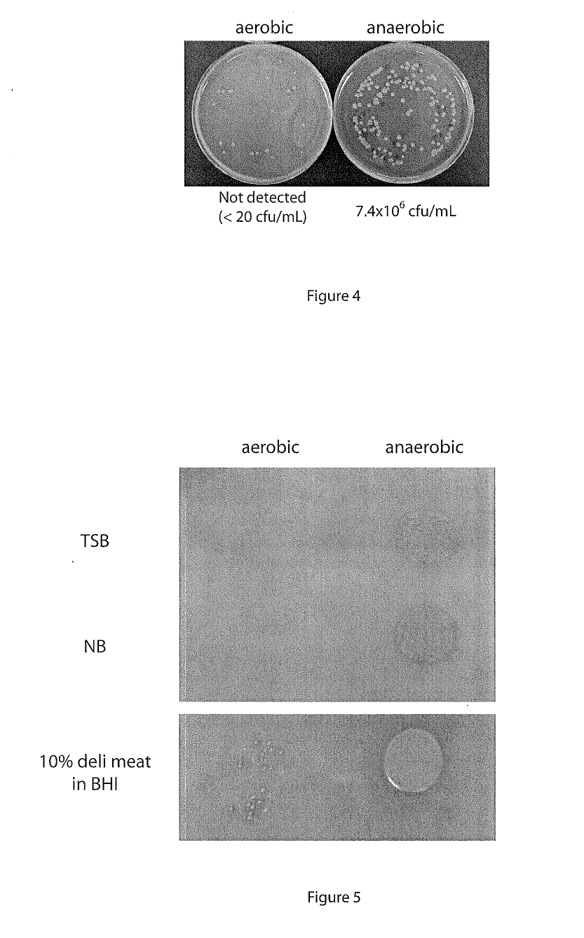

Two samples of 5 mL 10% ground beef homogenates were prepared by stomaching in Brain Heart Infusion Broth and inoculated with 7.3 cfu/mL L. monocytogenes 4b and 7.9×10⁴ cfu/mL E. coli O1:K1:H8. The samples were cultured in a rotational shaker (250 rpm) at 37° C. under aerobic and oxygen-poor conditions. The aerobic condition was established by allowing air ventilation. The oxygen-poor condition was established by preventing air ventilation and supplementing the culture with 20 μL/mL Oxyrase for broth (Oxyrase, OH). Dissolved oxygen concentrations were estimated at 8.7 mg/L for the aerobic condition and <0.01 mg/L for the oxygen-poor condition. After 24-hour culture, L. monocytogenes was detected and quantified by plate counting. As shown in FIG. 4, growth of L. monocytogenes was significantly promoted in the sample cultured under the oxygen-poor condition.

### Example 4

### Co-Cultures of L. monocytogenes and Competing Microorganisms

In two samples of 5 mL Tryptic Soy Broth (TSB), two samples of 5 mL Nutrient Broth (NB), and two samples of 5 mL 10% deli meat homogenate prepared by stomaching in Brain Heart Infusion Broth (BHI), each of which was inoculated with 7.4 cfu/mL L. monocytogenes 4b, 1.0 cfu/mL E. coli O157:H7 and 7.8×10⁴ cfu/mL E. coli O1:K1:H8, were investigated. The samples were cultured in a rotational shaker (250 rpm) at 37° C. under aerobic and oxygen-poor conditions. The aerobic condition was established by allowing air ventilation. The oxygen-poor condition was established by preventing air ventilation and supplementing 100 μL/mL Oxyrase for broth (Oxyrase, OH). Dissolved oxygen concentrations were estimated at 8.7 mg/L for the aerobic condition and <0.01 mg/L for the oxygen-poor condition. After 24-hour culture, L. monocytogenes was detected by chromogenic agar plate. As shown in FIG. 5, growth of L. monocytogenes was significantly promoted in the samples cultured under the oxygen-poor condition.

### Example 5

### Growth Enhancement of <10 cfu/mL L. monocytogenes in Co-Culture with More than 10⁴ cfu/mL of a Combination of Competing Microorganisms was Investigated

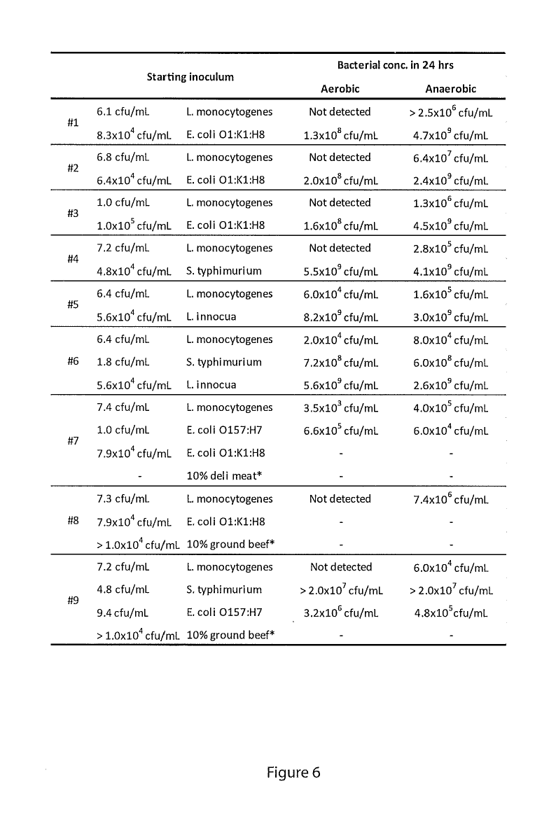

5 mL Brain Heart Infusion Broth or 10% food homogenates prepared by stomaching in Brain Heart Infusion Broth were inoculated with L. monocytogenes 4b, Listeria innocua (L. innocua), Salmonella typhimurium (S. typhimurium), E. coli O157:H7 or E. coli O1:K1:H8. The samples were cultured in a rotational shaker (250 rpm) at 37° C. under aerobic and oxygen-poor conditions. The aerobic condition was established by allowing air ventilation. The oxygen-poor condition was established by preventing air ventilation and supplementing Oxyrase for broth or Oxyrase for agar (Oxyrase, OH) at 20 or 100 μL/mL. Dissolved oxygen concentrations were estimated at 8.7 mg/L for the aerobic condition and <0.01 mg/L for the oxygen-poor condition, After 24-hour culture, L. monocytogenes 4b, L. innocua, S. typhimurium, E. coli O157:H7 or E. coli O1:K1:H8 was detected and quantified by plate counting. Growth of L. monocytogenes was significantly promoted by oxygen-poor culture and other food pathogens such as S. typhimurium and E. coli O157:H7 were also simultaneously detected (FIG. 6).

Any numbers expressing quantities of ingredients, constituents, reaction conditions, and so forth used in the specification are to be understood as being modified in all instances by the term “about”. Notwithstanding that the numerical ranges and parameters setting forth, the broad scope of the subject matter presented herein are approximations, the numerical values set forth are indicated as precisely as possible. Any numerical value, however, may inherently contain certain errors or inaccuracies as evident from the standard deviation found in their respective measurement techniques. None of the features recited herein should be interpreted as invoking 35 U.S.C. §112, ¶6, unless the term “means” is explicitly used.

Although the present invention has been described in connection with preferred embodiments thereof, it will be appreciated by those skilled in the art that additions, deletions, modifications, and substitutions not specifically described may be made without departing from the spirit and scope of the invention.

1. A culturing method to prevent the Jameson effect, comprising culturing a sample comprising at least one species of a target pathogen and competing microorganisms under an oxygen-poor condition in a growth medium.

2. The method of claim 1, wherein the competing microorganisms have faster growth rates and/or higher starting concentrations than the at least one target pathogen.

3. The method of claim 1, wherein the growth medium comprises a non-selective growth medium.

4. The method of claim 1, wherein the competing microorganisms have starting concentrations at least 100 times higher than the target pathogens.

5. The method of claim 1, wherein the target pathogens comprise one or more of: genus Listeria, genus Salmonella, genus Escherichia and genus Campylobacter.

6. The method of claim 1, further comprising conducting a detection assay to detect and/or quantify the at least one target pathogen.

7. The method of claim 1, wherein dissolved oxygen concentration of the growth medium is below 6.6 mg/L.

8. The method of claim 1, wherein dissolved oxygen concentration of the growth medium is below 3.3 mg/L.

9. The method of claim 1, wherein dissolved oxygen concentration of the growth medium is below 1.3 mg/L.

10. The method of claim 1, the growth medium is Brain Heart Infusion Broth, Nutrient Broth or Tryptic Soy Broth.

11. The method of claim 1, wherein a detection assay is used to detect and/or quantify the target pathogens, the detection assay comprising an agar plate, chromogenic agar plate, enzyme-linked immunosorbent assay (ELISA), immunochromatography, polymerase chain reaction (PCR), reverse transcription PCR(RT-PCR), real-time PCR, real-time RT-PCR, nucleic acid sequence based amplification (NASBA), loop-mediated isothermal amplification (LAMP), isothermal nucleic acid amplification, nucleic acid probe, biosensor, multiplex PCR, multiplex real-time PCR, DNA microarray, protein microarray or Luminex system.

12. The method of claim 1, wherein the sample has at least two species of target pathogens.

13. The method of claim 3, wherein the non-selective medium is free of antibiotics.

14. The method of claim 1, wherein the oxygen-poor condition is established by culturing the sample under an oxygen-poor atmosphere, supplementing the growth medium with an oxygen depletion agent, culturing the sample in a seal container, stationary culturing and/or applying a layer of oil on surface of the growth medium.

15. The method of claim 14, wherein the oxygen depletion agent is Oxyrase enzyme, alcohol oxidase, glucose oxidase, cysteine and/or titanium (III) citrate.

16. The method of claim 1, the culture medium is a liquid, semi-liquid or solid medium.

17. The method of claim 1, wherein the sample is located on a highly porous filter material, used to collect the target pathogens and the competing microorganisms from a large volume particulate sample.

18. The method of claim 1, wherein the at least one target pathogen is a bacteria of genus Listeria.

19. The method of claim 1, wherein the dissolved oxygen concentration of the growth medium is below 50% of atmospheric oxygen concentration.

20. The method of claim 19, wherein the dissolved oxygen concentration of the growth medium is below 25% of atmospheric oxygen concentration.

21. The method of claim 20, wherein the dissolved oxygen concentration of the growth medium is below 10% of atmospheric oxygen concentration.

22. A culturing method to prevent the Jameson effect, comprising culturing a sample comprising at least one target pathogen, and competing microorganisms with higher starting concentrations and/or faster growth rates than the at least one target pathogen, under an oxygen-poor condition in a growth medium, and conducting a detection assay to detect the presence and/or concentration of the at least one target pathogen.

23. The method of claim 22, wherein the competing microorganisms have faster growth rates and/or higher starting concentrations than the at least one target pathogen.

24. The method of claim 22, wherein the growth medium comprises a non-selective growth medium.

25. The method of claim 22, wherein the competing microorganisms have starting concentrations at least 100 times higher than the target pathogens.

26. The method of claim 22, wherein the target pathogens comprise one or more of: genus Listeria, genus Salmonella, genus Escherichia and genus Campylobacter.

27. The method of claim 22, wherein dissolved oxygen concentration of the growth medium is below 6.6 mg/L.

28. The method of claim 22, wherein dissolved oxygen concentration of the growth medium is below 3.3 mg/L.

29. The method of claim 22, wherein dissolved oxygen concentration of the growth medium is below 1.3 mg/L.

30. The method of claim 22, the growth medium is Brain Heart Infusion Broth, Nutrient Broth or Tryptic Soy Broth.

31. The method of claim 22, wherein the detection assay comprises an agar plate, chromogenic agar plate, enzyme-linked immunosorbent assay (ELISA), immunochromatography, polymerase chain reaction (PCR), reverse transcription PCR(RT-PCR), real-time PCR, real-time RT-PCR, nucleic acid sequence based amplification (NASBA), loop-mediated isothermal amplification (LAMP), isothermal nucleic acid amplification, nucleic acid probe, biosensor, multiplex PCR, multiplex real-time PCR, DNA microarray, protein microarray or Luminex system.

32. The method of claim 22, wherein the sample has at least two species of target pathogens.

33. The method of claim 22, wherein the growth medium is a non-selective medium.

34. The method of claim 33, wherein the non-selective medium is free of antibiotics.

35. The method of claim 22, wherein the oxygen-poor condition is established by culturing the sample under an oxygen-poor atmosphere, supplementing the growth medium with an oxygen depletion agent, culturing the sample in a seal container, stationary culturing and/or applying a layer of oil on surface of the growth medium.

36. The method of claim 35, wherein the oxygen depletion agent is Oxyrase enzyme, alcohol oxidase, glucose oxidase, cysteine and/or titanium (III) citrate.

37. The method of claim 22, the culture medium is a liquid, semi-liquid or solid medium.

38. The method of claim 22, wherein the sample is located on a highly porous filter material, used to collect the target pathogens and the competing microorganisms from a large volume particulate sample.

39. The method of claim 22, wherein the at least one target pathogen is a bacteria of genus Listeria.

40. The method of claim 22, wherein the dissolved oxygen concentration of the growth medium is below 50% of atmospheric oxygen concentration.

41. The method of claim 40, wherein the dissolved oxygen concentration of the growth medium is below 25% of atmospheric oxygen concentration.

42. The method of claim 41, wherein the dissolved oxygen concentration of the growth medium is below 10% of atmospheric oxygen concentration.
